(19) United States
(12) Patent Application Publication
(10) Pub. No.: US 2026/0301162 A1
(43) Pub. Date: Oct. 1, 2026

(54) Radiomic Texture Analysis of Transient Volumetric Fields

(71) Applicant: Tufts Medical Center, Inc., Boston, MA (US)

(72) Inventors: Adel M. Malek, Weston, MA (US); Alexandra Lauric, Medford, MA (US)

(21) Appl. No.: 19/095,199

(22) Filed: Mar. 31, 2025

(51) Int. Cl. G06T 7/00; G06T 7/40; G16H 50/50

(52) U.S. Cl. CPC G06T 7/0012; G06T 7/40; G16H 50/50; G06T 2207/10116; G06T 2207/20081; G06T 2207/30016; G06T 2207/30104

(57) ABSTRACT

A method for analyzing volumetric data includes using computational fluid dynamics to model fluid flow adjacent to a first surface, evaluating a physical quantity on the first surface, the physical quantity resulting from the flow, mapping the first surface to a second surface, the second surface surrounding first surface, mapping the second surface to a third surface, integrating the third surface into a volume, thereby defining the volumetric data, the volumetric data having at least one non-spatial coordinate, and carrying out texture analysis on pixels within the volumetric data. The mapping from the first surface to the second surface is continuous and the mapping from the second surface to the third surface is discontinuous along a one-dimensional subspace of the first surface.

### BACKGROUND

Aneurysms arise due to the localized weakening of a blood vessel wall, which, under sustained hemodynamic stress, can progressively expand. This pathological dilatation can remain stable or continue to enlarge until rupture occurs. The rupture of an aneurysm leads to severe clinical consequences, particularly in the case of intracranial aneurysms, where hemorrhagic events may result in significant neurological impairment or death.

Early detection and risk stratification of aneurysms are critical for clinical decision-making. Current imaging modalities allow visualization of aneurysms, and interventional procedures exist to mitigate rupture risk. However, due to the inherent risks associated with surgical interventions, it is imperative to accurately identify aneurysms at the highest risk of rupture to optimize treatment strategies.

### SUMMARY

An intact aneurysm has a wall exposed to pulsatile blood flow within its host blood vessel. This pulsatile blood flow in turn causes a time-varying shear stress on the aneurysm's wall. The magnitude and direction of this shear stress varies with location on the aneurysm's wall. This shear stress, which varies both spatially and temporally, is a factor that can be used to assess the likelihood of a rupture.

The introduction of temporal variation poses a challenge for known methods of analysis. The methods and systems described herein address this challenge through the incorporation of time variation in volumetric data results in a four-dimensional space in which there exist three spatial dimensions and one temporal dimension. These methods and systems can thus use the existing infrastructure of radiomic analysis to observe time-varying physical quantities that are relevant to analysis of the risk of a burst aneurysm, and in particular, to the risk of a burst in an intracranial aneurysm. These physical quantities include flow velocity and wall shear-stress on the dome of the aneurysm.

Cerebral aneurysm are degenerative processes resulting in pathological remodeling of the arterial wall based on the complex interaction between vessel wall biology and hemodynamic forces. This interaction is considered to be a major contributor to aneurysm formation and rupture.

Among hemodynamic features, wall shear-stress and features derived therefrom are of considerable significance. Evidence suggests that the wall shear-stress regulates the histological structure of arterial walls. As such, wall shear-stress is an important determinant of overall vascular structural remodeling.

The invention provides a way to leverage the existing infrastructure of radiomics to accommodate the analysis of time-varying volumetric data within, around, or on the surface of a morphologically irregular volume. The overall method has two distinct steps.

The first step is to map the surface of the morphologically irregular volume onto a morphologically regular form. A particularly useful way to achieve this is to project the volume onto a unit sphere.

The second step is to incorporate temporal information into the spatial domain. A useful way to carry this out is to form a four-dimensional data structure in which spatial information corresponding to a particular time is encoded plane that is transverse to a temporal axis. This allows conventional radiomics infrastructure to evaluate temporal characteristics in both the spatial and temporal domain at the same time.

In one aspect, the invention features a method that includes analyzing volumetric data by acquiring an image of a structure that is in a fluid, acquiring information indicative of time-varying mechanical energy applied to the fluid during a time interval, and using computational fluid dynamics to model fluid flow of the fluid flow adjacent to a first surface during the time interval, the first surface being defined based on the image of the structure. In such a method, the use of computational fluid dynamics to model the fluid flow includes, for each of a plurality of times in the time interval: evaluating a physical quantity on the first surface, the physical quantity resulting from the fluid flow, forming a first texture-map on the first surface, mapping the first texture-map to a second surface, thereby forming a second texture-map on the secund surface. The second surface is one that surrounds surrounding the first surface. The method continues with mapping the second texture-map from the second surface to a third surface, thereby forming a third texture-map on the third surface. The use of computational fluid dynamics to model the fluid flow results in a plurality of third texture-maps, each of which corresponds to values of the physical quantity at a corresponding one of the times in the plurality of times in the time interval. According to the method, the use of computational fluid dynamics to model the fluid flow further includes integrating the third texture-maps into a volume, thereby defining the volumetric data, the volumetric data having at least one non-spatial coordinate and carrying out texture analysis on pixels within the volumetric data, the texture analysis including analysis of evolution of texture during the time interval. The mapping from the first texture-map to the second surface is a continuous mapping and the mapping from the second texture-map to the third surface is discontinuous along a one-dimensional subspace of the first surface.

In some practices of the method, the second surface is a surface of a unit sphere.

In other practices of the method, the volume is a prism having a longitudinal axis, a first transverse axis, and a second transverse axis. In such practices, the longitudinal axis extends along a temporal direction, the first transverse axis extends along a first spatial direction, and the second axis extends along a second spatial direction.

Still other practices of the method include those in which the physical quantity includes shear stress on the first surface and those in which the physical quantity includes flow velocity at the first surface, those in which the physical quantity includes pressure at the first surface, those in which the physical quantity includes vorticity of fluid flow at the first surface, those in which the physical quantity includes helicity of fluid flow at the first surface, those in which the physical quantity includes the curl of a fluid flow field at the first surface, and those in which the physical quantity includes a volume integral of an inner product of the a fluid flow field at the first surface and a curl of the fluid flow field at the first surface.

Among the practices of the invention are those that include selecting the first surface to be a dome of an aneurysm, those that include selecting the first surface to be a wall of an intracranial aneurysm, and those that include selecting the volume to have units of square-meter seconds.

Practices include those in which the use of computational fluid dynamics to model fluid flow includes using it for simulating blood flow in a blood vessel and those in which
using computational fluid dynamics to model fluid flow includes using FSI to simulate interaction of blood in a blood vessel with the first surface, the first surface being a wall of an aneurysm.

Still other embodiments include those in which carrying out texture analysis comprise using radiomics to carry out the texture analysis, those in which carrying out texture analysis comprise using a machine-learning tool to carry out the texture analysis, and those in which carrying out texture analysis comprise using a deep-learning tool to carry out the texture analysis.

In another aspect, a method for analyzing volumetric and surface data, comprises simulating blood flow dynamics (such as using computational fluid dynamics (CFD), fluid-structure interaction (FSI), etc.) to model flow and structure behavior within diseased (such as harboring intracranial aneurysms) and non-diseased arterial vessels having a wall, evaluating volumetric parameters (such as volume, vorticity, helicity, pressure, displacement, etc.) and surface parameters (such as wall shear stress, pressure, tension etc.) resulting from the flow, and processing data taking into account parameter type (volumetric or surface) and non-spatial characteristics (such as evolution over time), including (1) processing volumetric data by projecting the input volume to a two-dimensional surface, integrating the second surface into a volume, thereby defining the volumetric data, the volumetric data having at least one non-spatial coordinate, using tools for feature extraction and feature analysis(such as radiomics, machine learning, deep learning, etc.), and carrying out analysis of the volumetric data, wherein the projection from the volume to the first surface preserves attenuation information and the projection stacking in a volumetric data captures pattern characteristics in the non-spatial domain, and (2) processing three-dimensional surface data of arbitrary shapes by mapping the wall parameters to a first surface, the first surface enclosing the region of interest (such as aneurysmal surface), mapping the first surface to a second surface, integrating the second surface into a volume, thereby defining the volumetric data, the volumetric data having at least one non-spatial coordinate, and using feature extraction and feature analysis tools, carrying out texture analysis on pixels within the volumetric data, wherein the mapping from the wall to the first surface is continuous, and wherein the mapping from the first surface to the second surface is a discontinuous unraveling of the first surface (3D) to the second surface (2D).

In another aspect, the invention features a method for analyzing volumetric and surface data, the method comprising simulating blood flow dynamics (such as using computational fluid dynamics (CFD), fluid-structure interaction (FSI), etc.) to model flow and structure behavior within diseased (such as harboring intracranial aneurysms) and non-diseased arterial vessels having a wall, evaluating volumetric parameters (such as volume, vorticity, helicity, pressure, displacement, etc.) and surface parameters (such as wall shear stress, pressure, tension etc.) resulting from the flow, and processing data taking into account parameter type (volumetric or surface) and non-spatial characteristics (such as evolution over time), (1) processing volumetric data by projecting the input volume onto a two-dimensional surface, integrating the second surface into a volume, thereby defining the volumetric data, the volumetric data having at least one non-spatial coordinate, and using radiomics, carrying out texture analysis on pixels within the volumetric data, wherein the projection from the volume to the first surface preserves attenuation information, and the projection stacking in a volumetric data captures pattern characteristics in the non-spatial domain, and (2) processing three-dimensional surface data of arbitrary shapes by mapping the wall parameters to a first surface, the first surface enclosing the region of interest (such as aneurysmal surface), mapping the first surface to a second surface, integrating the second surface into a volume, thereby defining the volumetric data, the volumetric data having at least one non-spatial coordinate, and using radiomics, carrying out texture analysis on pixels within the volumetric data, wherein the mapping from the wall to the first surface is continuous, and wherein the mapping from the first surface to the second surface is a discontinuous unraveling of the first surface (3D) to the second surface (2D).

In another aspect, the invention features a method for analyzing volumetric and surface data associated with a vessel, the vessel being an arterial vessel. Such a method includes simulating dynamics of blood flow to model flow and structural behavior within the vessel, evaluating volumetric parameters and surface parameters resulting from the blood flow, and processing data taking into account parameter type and non-spatial characteristics. In such a method, processing the data includes processing volumetric data, the volumetric data including a non-spatial coordinate. Processing the volumetric data, in turn, includes projecting an input volume onto each surface from a plurality of surfaces and doing so in a manner that preserves attenuation information, each of the surfaces being a two-dimensional surface. The method further includes using a tool to analyze the volumetric data, the tool being selected from the group consisting of a feature-extraction tool and a feature-analysis tool, stacking the surfaces onto which the input volume has been projected, thereby capturing evolution of pattern characteristics in the non-spatial coordinate, and processing surface data on a surface of an arbitrary shape. Processing the surface data includes mapping the wall parameters to a first surface, the first surface enclosing a region of interest, mapping the first surface onto a second surface, integrating the second surface into a volume, thereby defining the volumetric data, the volumetric data having at least one non-spatial coordinate, and using feature extraction and feature analysis tools, carrying out texture analysis on pixels within the volumetric data. According to the method, the mapping from the wall to the first surface is continuous and the mapping from the first surface to the second surface is a discontinuous unraveling of the first surface to the second surface.

Practices of the method include those in which simulating the blood flow dynamics includes using computational fluid dynamics to simulate the blood flow and those in which simulating the blood flow dynamics includes carrying out a simulation using FSI to simulate interaction of blood with the artery.

Still other practices include those in which the arterial vessels comprise at least one arterial vessel that harbors an intracranial aneurysm.

Among the practices are those in which selecting the volumetric parameters includes selecting one of volume, vorticity, helicity, pressure, and displacement as the volumetric parameter. Still other practices include those in which evaluating the volumetric and surface parameters includes evaluating at least one of wall shear stress, pressure, and tension.

Still other practices include those in which processing the data taking into account the parameter type and non-spatial characteristics includes taking into account at least one of a volumetric parameter and a surface parameter and taking into account of evolution over time.

Further practices are those in which using the tool includes using a radiomics tool, a machine-learning tool, or a deep-learning tool.

Still other practices include those in which the region-of-interest includes an aneurysmal surface.

In another aspect, the invention features an apparatus for analyzing volumetric data. Such an apparatus includes radiomics circuitry and image acquisition circuitry. The image acquisition circuitry provides, to the radiomics circuitry, an image of a first surface that is in a fluid. The radiomics circuitry includes a flow simulator, first and second projectors, and an assembler. The flow simulator is configured: to receive both the image and information indicative of time-varying mechanical energy applied to the fluid during a time interval that includes a plurality of times, to use computational fluid dynamics to model flow of the fluid adjacent to the first surface during the time interval, and to generate, for each of the times in the plurality of times, a corresponding first texture-map on the first surface, the first texture-map being indicative of a physical quantity resulting from the fluid flow. The first projector is configured to carry out a continuous mapping of each of the first texture-maps to a second surface that surrounds the first surface, whereby the first projector generates a plurality of second texture-maps, each of which is a mapping onto the second surface at a corresponding time from the plurality of times in the time interval. The second projector is configured to carry out a discontinuous mapping of each of the second texture-maps to a third surface, the third surface being discontinuous along a one-dimensional subspace of the first surface, whereby the second projector generates a plurality of third texture-maps, each of which is a mapping onto the third surface at a corresponding one of the times in the time interval. The assembler is configured to integrate the third texture-maps into a volume, thereby defining the volumetric data, the volumetric data having at least one non-spatial coordinate.

The foregoing subject matter is described herein in the context of aneurysms, and in particular, using fluid-flow simulation data that resulted from an analysis of intracranial aneurysms using methods of computational fluid dynamics to simulate blood flow within the aneurysm and its surrounding vasculature during the course of a cardiac cycle. This resulted in collection of data in the spatial domain. Such data represents a physical quantity characterized by a two-dimensional field, such as shear stress on the aneurysm's wall, pressure on the aneurysm's surface, or one characterized by a three-dimensional field, such as such as flow velocity, vorticity, or helicity in the flow domain.

The technique is directly applicable to such non-abstract and practical applications as discriminating between ruptured and unruptured aneurysms. Since humans are known to have died as a result of a ruptured aneurysm, the technique described herein has real-world applications of considerable practical value. The method and system described herein thus extends the use of existing radiomics infrastructure to evaluate the evolution of a physical quantity during the entire cardiac cycle.

The techniques described herein are described in the context of biomedical applications, and in particular, in the context of the effects of transient fluid flow on aneurysms. However, the techniques are broadly applicable to the analysis of other phenomena in which volumetric data varies over time. Examples include weather simulations, seismic data, data related to environmental monitoring, such as ocean currents, forest growth, and dispersion of matter, such as pollutants. Other examples include fluid flow simulation, astronomical data, traffic data, and econometric data. In all these fields, known techniques tend to focus predominantly on one domain (i.e., spatial or temporal) to the detriment of the other.

It has been discovered through experimentation that the methods and systems described herein cannot be implemented using a generic computer. Accordingly, any computer that implements the methods or systems disclosed herein is a non-generic computer.

It has also been discovered that the methods and systems described herein cannot practicably be performed in the human mind whether alone or augmented with a writing utensil and a substrate that accepts writings from such a writing utensil.

The appended claims are deemed to cover only non-abstract implementations and systems. As used herein, “non-abstract” shall mean the converse of “abstract” as that term has been defined by the courts of the United States as of the filing date of this application. Any person who construes the claims otherwise shall be deemed to be construing the claims contrary to the specification.

These and other features of the invention will be apparent from the following detailed description and the accompanying figures, in which:

### DESCRIPTION OF DRAWINGS

FIG. 1 shows radiomic circuitry;

FIG. 2 shows an alternative embodiment of the radiomic circuitry show in in FIG. 1.

FIG. 3 shows texture maps produced by the radiomic circuitry of FIG. 1 after having been arranged into a volume having a temporal axis and spatial axes; and

FIG. 4 shows a process carried out by the radiomic circuitry of FIG. 1.

### DESCRIPTION

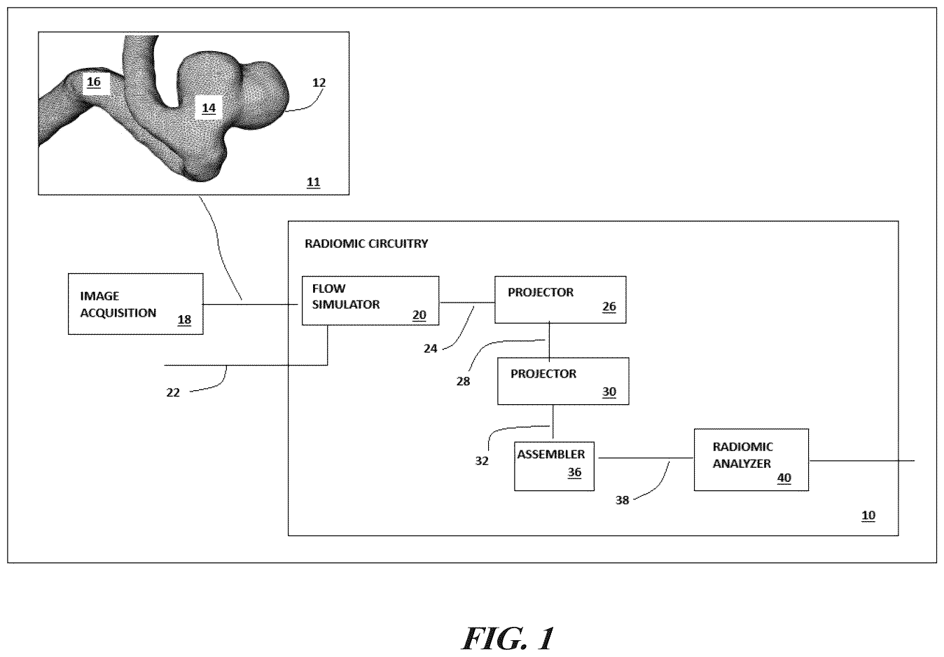

FIG. 1 shows radiomic circuitry 10 for analysis of a time-varying physical quantity that arises as a result of flow past the wall 12 of a structure 14. In the particular embodiment described herein, the structure is an aneurysm 14 that has formed in a blood vessel 16 and the flow in question is the flow of blood past the wall 12 of this aneurysm 14 or in the vicinity of this wall 12

Examples of a physical quantity that is of interest under such circumstances include the flow velocity at the wall 12 and the shear stress at the wall 12. Other examples of a physical quantity that is of interest include the flow velocity within the volume that is in the vicinity of the aneurysm 14. In both cases, the physical quantity defines a field that, for each point in a two-dimensional subspace, has a value.

Image-acquisition circuitry 18 provides the radiomic circuitry 10 with an image 11 of the blood vessel's interior volume, including the geometry of the aneurysm 14.

Examples of an image 11 provided by the image-acquisition circuitry 18 are those acquired through such conventional methods as MRI, PET, and CT scanning, and angiography. For analysis of an aneurysm 14, an image 11 obtained using 3D catheter angiography is particularly useful.

Existence of an aneurysm 14 gives rise to a risk of its rupture. A practical application of the radiomic circuitry 10 is therefore that of providing an objective method for assessing that risk so that important medical and lifestyle decisions can be made. It has been discovered that, in an effort to assess this risk, it is useful to observe the interaction of the wall 12 of the aneurysm 14 with the forces that result from the pulsatile blood-flow present in the blood vessel 16 during a cardiac cycle. This pulsatile blood-flow generates a time-varying stress field on the wall 12 of the aneurysm 14.

To assess this effect, the radiomic circuitry 10 features a flow-simulation circuit 20. The flow-simulation circuit 20 receives a cardiac-cycle input 22 representative of a cardiac cycle and an image 11 from the image-acquisition circuitry 18. Using the cardiac-cycle input 22 and the geometry defined by the image 11 from the image-acquisition circuitry 18, the flow-simulation circuit 20 applies known techniques from computational fluid dynamics to evaluate a time-varying flow field in the neighborhood of the aneurysm 14. This time-varying flow field interacts with the wall 12 to form a time-varying shear-stress field on the aneurysm's wall 12 and its evolution during the course of the cardiac cycle.

In the embodiment shown in FIG. 1, the flow simulator 20 receives the image 11 directly. In such cases, the flow simulator 20 evaluates the field along a surface that corresponds to a structure within the image 11, such as the aneurysm's wall 12.

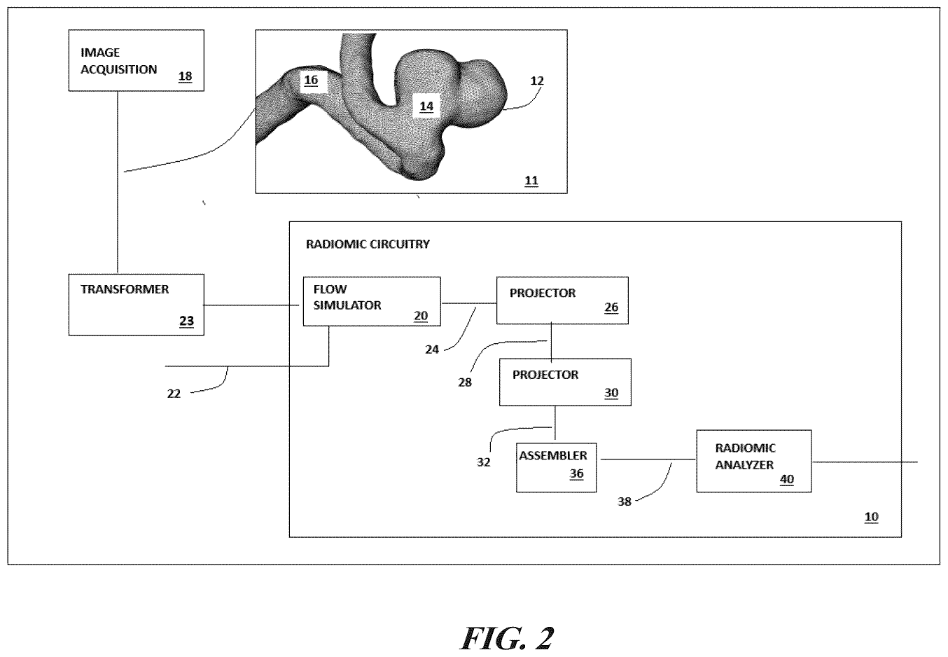

In other embodiments, such as that shown in FIG. 2, there exists a transformer 23 that transforms the image 11. This provides a way to cause the flow simulator 20 to evaluate the field along a surface other than that defined by the aneurysm's wall 12.

Among these embodiments are those in which the transformer 23 generates a centroid-radii model of the wall 12, which it then provides to the flow simulator 20 for further processing. In particular, the flow simulator 20 evaluates the field along the surface of the centroid-radii model.

In other embodiments, the transformer 23 defines a two-dimensional subspace within the volume as being the surface and provides that surface to the flow simulator 20 for further processing. The flow simulator 20 then evaluates the field along the surface defined by this two-dimensional subspace.

A first texture-map 24 represents the shear-stress field at a particular instant during the cardiac cycle. The result of the process carried out by the flow-simulation circuit 20 over the course of a cardiac cycle is thus a set of first texture-maps 24, each of which corresponds to the shear-stress field at a particular instant of time during the cardiac cycle. This set of first texture-maps 24 thus provides information on the evolution of the shear-stress field on the aneurysm's wall 12 during the course of a complete cardiac cycle.

In some embodiments, the two-dimensional subspace corresponds to the aneurysm's dome. In such embodiments, each of the first texture-maps 24 shows values of a field that is defined on the aneurysm's dome. In some embodiments, the function represents the shear stress on the dome as a function of position thereon. In others, it represents the flow velocity.

The aneurysm's dome is an irregularly-shaped two-dimensional subspace of the volume defined by the blood vessel's interior. As a result of its irregularity, this two-dimensional subspace is not directly amenable to radiomic analysis.

To address the foregoing difficulty, each of the first texture-maps 24 is provided to a first projector 26. This first projector 26 projects each of the first texture-maps 24 onto a unit sphere to form a second texture-map 28. The first projector 26 thus alleviates the difficulty caused by having a dome of irregular shape. However, the set of second texture maps 28 remains inconvenient for analysis of the evolution of shear stress during the course of a cardiac cycle.

To address this remaining inconvenience, each of the second texture-maps 28 is provided to a second projector 30. The second projector 30 projects each of the second texture-maps 28 onto a corresponding plane. This results in a set of third texture-maps 32, each of which corresponds to an instant of time during the cardiac cycle.

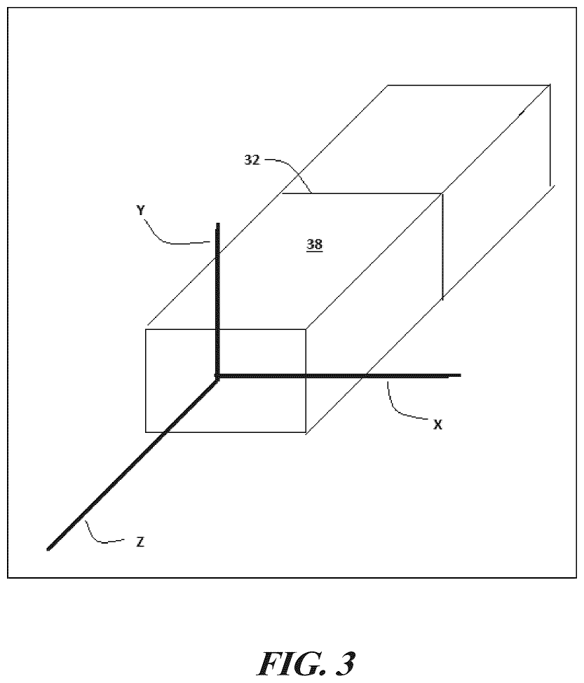

In contrast to the second texture-maps 28, the third texture-maps 32 are easily arrangeable to form a volume with one temporal dimension and two spatial dimensions. Assuming that the third texture-maps 32 all have the same size, the resulting volume is a prism 34, as shown in FIG. 3. The longitudinal axis “Z” of the prism 34 corresponds to a temporal coordinate. Its transverse axes “X,” “Y” correspond to spatial coordinates. The prism 34 is thus a space-time prism 34.

This set of third texture-maps 32 is then provided to an assembler 36, the output of which an ordered prism 38 of third texture-maps 32 but arranged in the correct temporal order. This ordered space-time prism 38 is a convenient input for a radiomic analyzer 40 that considers not only the spatial dimension, i.e., the aneurysm's dome, but also the temporal dimension. The resulting ordered space-time prism 38 that reveals the evolution of shear stress on the dome during the course of a cardiac cycle.

The use of the radiomic analyzer 40 in the evaluation of transient flow data offers a novel perspective on the understanding of aneurysmal rupture. The radiomic analyzer 40 provides an effective way to discriminate the ruptured status of aneurysms based on images of shear stress on the aneurysm's wall 12. These valuable insights that might otherwise go unnoticed using traditional processing tools.

The processing steps described herein extend the use of radiomics beyond angiographic analysis and into the regime of evaluating the evolution of shear stress on an aneurysm's dome during the course of a cardiac cycle, not only in terms of distribution, but also texture and imaging patterns.

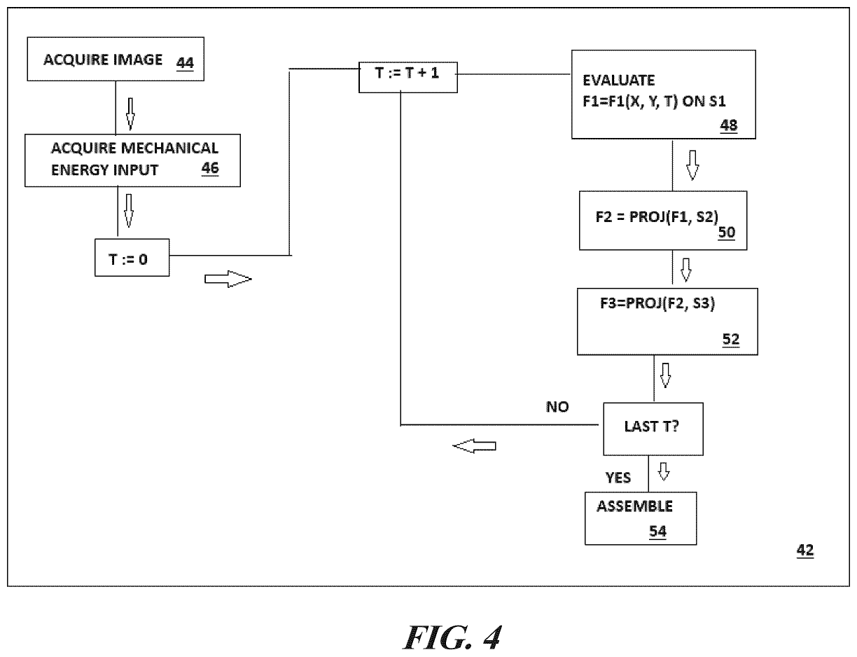

FIG. 4 shows a method 42 as carried out by the radiomic circuitry 10.

The method 42 begins with the acquisition of the image 11 of a wall 12 that is in a fluid (step 44) and acquisition of an input 22 that provides information about mechanical energy applied to the fluid during a time interval (step 46). Then, for each time from a plurality of times within the time interval, the method 42 continues with evaluating the physical quantity over a first surface (step 48), thus forming a first texture-map 24, projecting the first texture-map 24 onto a second surface (step 50), thus forming a second texture-map 28, and then then projecting the second texture-map 28 onto a third surface (step 52) to form a third texture-map 32. After having carried out these steps for all the relevant times, the method 42 includes assembling the third texture-maps 32 into temporal order to construct an ordered prism 38 having two spatial dimensions and one temporal dimension (step 54).

What is claimed is:

1. A method comprising analyzing volumetric data, wherein analyzing said volumetric data comprises:
acquiring an image of a structure that is in a fluid,
acquiring information indicative of time-varying mechanical energy applied to said fluid during a time interval,
using computational fluid dynamics to model fluid flow of said fluid adjacent to a first surface during said time interval, said first surface being based on said image of said structure,
wherein using said computational fluid dynamics to model said fluid flow comprises, for each of a plurality of times in said time interval:
evaluating a physical quantity on said first surface, said physical quantity resulting from said fluid flow,
forming a first texture-map on said first surface,
mapping said first texture-map to a second surface, thereby forming a second texture-map on said second surface, said second surface surrounding said first surface, and
mapping said second texture-map from said second surface to a third surface, thereby forming a third texture-map on said third surface,
whereby using said computational fluid dynamics to model said fluid flow results in a plurality of third texture-maps, each of which corresponds to values of said physical quantity at a corresponding one of said times in said plurality of times in said time interval,
wherein using said computational fluid dynamics to model said fluid flow further comprises:
integrating said third texture-maps into a volume, thereby defining said volumetric data, said volumetric data having at least one non-spatial coordinate and
carrying out texture analysis on pixels within said volumetric data, said texture analysis including analysis of evolution of texture during said time interval and
wherein said mapping from said first texture-map to said second surface is a continuous mapping and
wherein said mapping said second texture-map to said third surface is discontinuous along a one-dimensional subspace of said first surface.

2. The method of claim 1, wherein said second surface is a surface of a unit sphere.

3. The method of claim 1, wherein said volume is a prism having a longitudinal axis, a first transverse axis, and a second transverse axis, wherein said longitudinal axis extends along a temporal direction, wherein said first transverse axis extends along a first spatial direction, and wherein said second axis extends along a second spatial direction.

4. The method of claim 1, wherein said physical quantity comprises shear stress on said first surface.

5. The method of claim 1, wherein said physical quantity comprises flow velocity at said first surface.

6. The method of claim 1, further comprising selecting said first surface to be a dome of an aneurysm.

7. The method of claim 1, further comprising selecting said first surface to be a wall of an intracranial aneurysm.

8. The method of claim 1, further comprising selecting said volume to have units of square-meter seconds.

9. The method of claim 1, wherein using computational fluid dynamics to model fluid flow comprises simulating blood flow in a blood vessel.

10. The method of claim 1, wherein using computational fluid dynamics to model fluid flow comprises using FSI to simulate interaction of blood in a blood vessel with said first surface, said first surface being a wall of an aneurysm.

11. The method of claim 1, wherein said physical quantity comprises pressure at said first surface.

12. The method of claim 1, wherein said physical quantity comprises vorticity of fluid flow at said first surface.

13. The method of claim 1, wherein said physical quantity comprises helicity of fluid flow at said first surface.

14. The method of claim 1, wherein said physical quantity comprises the curl of a fluid flow field at said first surface.

15. The method of claim 1, wherein said physical quantity comprises a volume integral of an inner product of a fluid flow field at said first surface and a curl of said fluid flow field at said first surface.

16. The method of claim 1, wherein carrying out texture analysis comprise using radiomics to carry out said texture analysis.

17. The method of claim 1, wherein carrying out texture analysis comprise using a machine-learning tool to carry out said texture analysis.

18. The method of claim 1, wherein carrying out texture analysis comprise using a deep-learning tool to carry out said texture analysis.

19. A method for analyzing volumetric and surface data associated with a vessel, said vessel being an arterial vessel, said method comprising:
simulating dynamics of blood flow to model flow and structure behavior within said vessel,
evaluating volumetric parameters and surface parameters resulting from said blood flow, and
processing data taking into account parameter type and non-spatial characteristics,
wherein processing said data comprises processing volumetric data, said volumetric data comprising a non-spatial coordinate,
wherein processing said volumetric data comprises projecting an input volume onto each surface from a plurality of surfaces and doing so in a manner that preserves attenuation information, each of said surfaces being a two-dimensional surface,
using a tool to analyze said volumetric data, said tool being selected from the group consisting of a feature-extraction tool and a feature-analysis tool,
stacking said surfaces onto which said input volume has been projected, thereby capturing evolution of pattern characteristics in said non-spatial coordinate, and
processing surface data on a surface of an arbitrary shape, wherein processing said surface data comprises:
mapping the wall parameters to a first surface, the first surface enclosing a region of interest,
mapping the first surface onto a second surface,
integrating the second surface into a volume, thereby defining the volumetric data, the volumetric data having at least one non-spatial coordinate, and
using feature extraction and feature analysis tools, carrying out texture analysis on pixels within the volumetric data,
wherein the mapping from the wall to the first surface is continuous, and
wherein the mapping from the first surface to the second surface is a discontinuous unraveling of the first surface to the second surface.

20. The method of claim 19, wherein simulating said blood flow dynamics comprises using computational fluid dynamics to simulate said blood flow.

21. The method of claim 19, wherein simulating said blood flow dynamics comprises carrying out a simulation using FSI to simulate interaction of blood with said artery.

22. The method of claim 19, wherein said arterial vessels comprise at least one arterial vessel that harbors an intracranial aneurysm.

23. The method of claim 19, wherein evaluating volumetric parameters comprises selecting said volumetric parameters selected from the group consisting of volume, vorticity, helicity, pressure, and displacement.

24. The method of claim 19, wherein evaluating said volumetric and surface parameters comprises evaluating at least one of wall shear stress, pressure, and tension.

25. The method of claim 19, wherein processing said data taking into account said parameter type and non-spatial characteristics comprises taking into account at least one of a volumetric parameter and a surface parameter and taking into account of evolution over time.

26. The method of claim 19, wherein using said tool comprises using a tool selected from the group consisting of a radiomics tool, a machine-learning tool, and a deep-learning tool.

27. The method of claim 19, wherein said region-of-interest comprises an aneurysmal surface.

28. An apparatus for analyzing volumetric data, said apparatus comprising radiomics circuitry and image acquisition circuitry,
wherein said image acquisition circuitry provides, to said radiomics circuitry, an image of a structure that is in a fluid,
wherein said radiomics circuitry comprises a flow simulator, first and second projectors, and an assembler,
wherein said flow simulator is configured:
to receive first information and second information, wherein said first information is based on said structure and wherein said second information is indicative of time-varying mechanical energy applied to said fluid during a time interval that comprises a plurality of times,
to use computational fluid dynamics to model flow of said fluid adjacent to a first surface during said time interval, said first surface being defined based on said first information, and
to generate, for each of said times in said plurality of times, a corresponding first texture-map on said first surface, said first texture-map being indicative of a physical quantity resulting from said fluid flow,
wherein said first projector is configured to carry out a continuous mapping of each of said first texture-maps to a second surface that surrounds said first surface, whereby said first projector generates a plurality of second texture-maps, each of which is a mapping onto said second surface at a corresponding time from said plurality of times in said time interval,
wherein said second projector is configured to carry out a discontinuous mapping of each of said second texture-maps to a third surface, said third surface being discontinuous along a one-dimensional subspace of said first surface, whereby said second projector generates a plurality of third texture-maps, each of which is a mapping onto said third surface at a corresponding one of said times in said time interval, and
wherein said assembler is configured to integrate said third texture-maps into a volume, thereby defining said volumetric data, said volumetric data having at least one non-spatial coordinate.

29. The apparatus of claim 28, wherein said first information defines a surface of said structure as said first surface.

30. The apparatus of claim 28, wherein said first information defines a surface of a centroid radii model of said structure as said first surface 31. The apparatus of claim 28, wherein said first information defines a two-dimensional subspace of within a volume adjacent to said structure as said first surface.
